SEQUENCE LISTING (6 sequences)

SEQ ID NO: 1 - DNA, 21 bp, Mus musculus
cttccgcgccgatttcacctg

SEQ ID NO: 2 - DNA, 20 bp, Mus musculus
acccctcagactgtgtgagt

SEQ ID NO: 3 - DNA, 26 bp, Mus musculus
ctagctagatgaccctgcaccaccaa

SEQ ID NO: 4 - DNA, 27 bp, Mus musculus
ttgcctgtgcggcaggggttgggacga

SEQ ID NO: 5 - DNA, 24 bp, Mus musculus
cgcgctggcgcagacgcggttaaa

SEQ ID NO: 6 - DNA, 24 bp, Mus musculus
ctgctgattggccatgtgcgctca

(19) United States
(12) Patent Application Publication
(10) Pub. No.: US 2008/0031968 A1
(43) Pub. Date: Feb. 7, 2008

(54) Methods for increasing cellular energy expenditure

(75) Inventors: Antonio C. Bianco, Chestnut Hill, MA (US); Johan Auwerx, Hindisheim (FR); Marcelo A. Christoffolete, Brighton, MA (US); Sander Houten, Strasbourg (FR); Brian W. Kim, Boston, MA (US); Philip Reed Larsen, Brookline, MA (US); Mitsuhiro Watanabe, Illkirch-Graffenstadten (FR)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 11/392,914

(22) Filed: Mar. 30, 2006

Related U.S. Application Data
(60) Provisional application No. 60/667,057, filed on Apr. 1, 2005.
(60) Provisional application No. 60/672,172, filed on Apr. 18, 2005.

(51) Int. Cl. A61K 35/00; C12Q 1/18; G01N 33/53; G01N 33/567

(52) U.S. Cl. 424/528; 435/32; 435/7.1; 435/7.21

(57) ABSTRACT

The present invention is directed to methods of treating a human for a variety of conditions by administering an agonist of the G protein coupled receptor TGR5. In addition, the invention includes methods for determining whether a test compound is likely to be effective in treating one of these conditions by assaying it for its ability to raise intracellular iodothyronine deiodinase levels or for its ability to bind to and activate TGR5.

### CROSS REFERENCE TO RELATED APPLICATIONS

The present application claims priority to, and the benefit of, U.S. provisional applications 60/667,057 filed on Apr. 1, 2005 and 60/672,172, filed on Apr. 18, 2005. The contents of these prior applications are hereby incorporated by reference in their entirety.

### STATEMENT OF GOVERNMENT FUNDING

The United States Government has a paid-up license in this invention and the right in limited circumstances to require the patent owner to license others under reasonable terms as provided for by the terms of NIH Grant Nos. DK58538 and DK36256, awarded by the Department of Health and Human Services.

### FIELD OF THE INVENTION

The present invention is in the field of treatment methods that involve increasing energy expenditure by cells. It is particularly concerned with therapies involving the administration of bile acids and other agonists of the TGR5 receptor for the treatment of conditions such as hypothyroidism; hypertriglyceridemia; and thyroid dysfuinction. In addition, the invention is concerned with assays that can be used for identifying new compounds that may be used as therapeutic agents.

### BACKGROUND OF THE INVENTION

Bile acids (BAs) have long been known to play an essential role in dietary lipid absorption and cholesterol catabolism. In recent years, an important new role for BAs as signaling molecules has also emerged. Three major signaling mechanisms have been identified. First, BAs activate mitogen activated protein kinase (MAPK) pathways like the extracellular-regulated kinases (ERK) and the c-Jun NH2-terminal kinase (JNK) pathways (Gupta, et al., J. Biol. Chem. 276:15816 (2001); Qiao et al., Mol Cell Biol 23:3052 (2003)). Second, BAs are ligands for the G-protein coupled receptor (GPCR) TGR5 (also named BG37 or M-BAR) (Kawamata, et al., J. Biol. Chem. 278:9435 (2003); Maruyama, et al., Biochem. Biophys. Res. Commun. 298:714 (2002)), typically inducing adenylate cyclase activity and consequently the levels of cyclic adenosine monophospate (cAMP). Finally, conjugated and unconjugated BAs are the natural ligands that activate the nuclear hormone receptor farnesoid X receptor α (FXRα, NR1H4) (Makishima et al., Science 284:1362 (1999); Parks, et al., Science 284:1365 (1999); Wang, et al., Mol Cell 3:543 (1999)).

The discovery of BAs as the endogenous FXRα ligands suggested a role for this nuclear receptor in the regulation of the enterohepatic recycling of BAs and the feedback regulation of BA biosynthesis. Indeed, FXRα controls many genes involved in these processes (Houten, et al., Ann. Med. 36:482 (2004)). Most interestingly is the regulation of the short heterodimer partner (SHP, NR0B2) (Goodwin et al., Mol Cell 6:517 (2000); Lu, et al., Mol Cell 6:507 (2000)), an atypical nuclear hormone receptor that inhibits the activity of several other nuclear receptors. The FXRα-mediated SHP induction underlies the negative. feedback regulation of BA biosynthesis (Goodwin, et al., Mol Cell 6:517 (2000); Lu et al., Mol. Cell 6:507 (2000); Sinal, et al., Cell 102:731 (2000); Brendel, et al., Mol. Endocrinol. 16:2065 (2002); Kerr, et al., Dev. Cell 2:713 (2002); Wang et al., Dev. Cell 2:721 (2002)). FXRα inhibits BA synthesis also by regulation of the expression of fibroblast growth factor-19 (FGF-19). This secreted growth factor signals through the cell-surface FGF receptor-4 (FGFR-4) tyrosine kinase, resulting in an alternative pathway that potently suppresses BA biosynthesis in a SHP-independent fashion (Holt, et al., Genes Dev. 17:1581 (2003); Watanabe, et al., J. Clin. Invest. 113:1408 (2004)).

Although studies performed on mice have indicated that bile acids may be useful in treating or preventing obesity, the relevance of these studies to humans has remained uncertain. This is due in part to the fact that many of the effects seen in mice appear to be due primarily to the activation of brown adipose tissue which is essentially absent in adult humans. In addition, the exact mechanism by which bile acids exert their effects in obesity has remained uncertain. The discovery of this mechanism could lead to new treatment methods or suggest additional conditions where the administration of bile acids or other factors might be beneficial.

### SUMMARY OF THE INVENTION

The present invention is based upon experiments that demonstrate that bile acids prevent weight gain and lead to weight loss by activating type-2 iodothyronine deiodinase (D2). Results suggest that the activation of D2 and other factors by bile acids leads to increased cellular energy expenditure and improved metabolic control. The D2 enzyme is present in human skeletal muscle, and has been found to be activated when bile acids bind to the G-protein-coupled receptor TGR5, located on the membranes of muscle cells. Thus, the positive therapeutic effects of bile acids are mediated by TGR5 and other agonists for this receptor should have similar therapeutic properties. In particular such agonists should be useful in enhancing the expenditure of energy by tissues that co-express TGR5 and D2, i.e., brain, heart, skeletal muscle and, particularly, testes. Diseases or conditions that can benefit by increasing energy expenditure in one or more of these tissues should all be amenable to treatment TGR5 agonists. In addition, the invention is directed to assays examining the ability of agents to bind to TGR5 and activate D2 and which may be used to identify new agents of potential therapeutic value.

In its first aspect, the invention is directed to a method of treating a human for a variety of diseases or conditions by administering a therapeutically effective amount of an agonist of the receptor TGR5. The conditions that should respond to such treatment include: hypothyroidism; hypertriglyceridemia; thyroid dysfunction; resistance to thyroid hormone; low T3 syndrome; Wilson's syndrome; depression; attention deficit disorder; insulin resistance occurring with or without diabetes or obesity; glucose intolerance occurring with or without diabetes or obesity; hypertension; infertility; cardiac insufficiency; and syndromes or conditions that include mitochondrial dysfunction such as: Alzheimer's disease, Parkinson's disease, autism, and the aging process. The term, “therapeutically effective amount” means that sufficient agonist is administered to obtain an improvement with respect to a pathological characteristic of the disease, e.g., improved thyroid function, reduced insulin resistance, reduced glucose intolerance, reduced blood pressure, etc. The amount should be sufficient to increase intracellular levels of cAMP in skeletal muscle cells and to elevate the activity of type-2 iodothyronine deiodinase (D2). Preferred agonists are bile acids, with cholic acid (CA); chenodeoxycholic acid; and tauroCA being especially preferred. The final dosage should generally be 0.1-25 mg/kg body weight/day and preferably, 0.5-20 mg/kg body weight/day. In addition the agonists described in WO-2004067008 (hereby incorporated by reference) may be made and used.

In a second aspect, the invention is directed to a method of assaying a test compound for use in the treatment of one or more of the conditions described above. The method involves incubating cells that express D2 in the presence of the test compound, measuring either the amount of D2 expressed or the activity of the D2 enzyme in the incubated cells, and then comparing this activity with the activity or amount determined for cells assayed under similar conditions but in the absence of the test compound. It may be concluded that the test compound should be useful in the treatment methods if the activity of D2 is higher in the presence of the test compound than in its absence.

The invention also includes methods of determining whether a test compound may be therapeutically useful by assaying the compound for its ability to bind to and activate the TGR5 receptor, i.e., by determining if the compound acts as a TGR5 agonist. Binding may be determined using standard radioreceptor assays and activation can be assessed based upon any intracellular activity known to be induced as the result of TGR binding to a ligand such as cholic acid. Preferably, the intracellular activity followed is adenyl cyclase activity and/or D2 activity.

### DETAILED DESCRIPTION OF THE INVENTION

The present invention is based upon studies examining the effects of bile acids or salts of bile acids on triglyceride levels and energy homeostasis. The administration of cholic acid prevents hepatic triglyceride accumulation, very low density lipoprotein (VLDL) secretion, and elevated serum triglyceride levels in mouse models of hypertriglyceridemia. In a model of diet-induced obesity, administration of cholic acid or chenodeoxycholic acid prevents and reverses weight gain, decreases lipid storage in adipose tissue, induces energy expenditure, and improves metabolic control as shown by an increased insulin sensitivity.

At the molecular level in the liver, cholic acid decreases expression of sterol regulatory element-binding protein 1c (SREBP-1c) and its lipogenic target genes. The effects of bile salts on energy homeostasis in mice are mediated by brown adipose tissue (BAT). In this tissue, bile salts induce the expression of type 2 iodothyronine deiodinase (D2) and peroxisome proliferator-activated receptor γ coactivator-1. Results suggest that bile salts are mediators in the regulation of hepatic lipogenesis and adaptive thermogenesis. Although adult humans do not have substantial amounts of BAT, D2 is prevalent in certain tissues, e.g., skeletal muscle, and activated by the binding of bile acids to the TGR5 receptor.

A. Bile Acids and Salts of Bile Acids

The treatment methods described herein are directed, inter alia, to the administration of bile acids or other TGR5 agonists to individuals as a treatment for a variety of diseases. Any type of pharmaceutically acceptable bile acid or agonist that binds to TGR5 may be used for this purpose including all of the commonly known forms of cholic acid, and chenodeoxycholic acid. These compounds have been thoroughly studied in the art, and methods for their preparation are well known. Some of the more common bile acids are commercially available. It will be understood the term “bile acid” or “bile acids” as used herein includes not only the acids themselves but also pharmaceutically acceptable salts of these acids. Common derivatives and conjugates of the bile acids may also be successfully used in the treatment methods. Other TGR5 agonists may be made using methods known in the art (see, e.g., WO-2004067008).

B. Route of Administration

The methods and compositions discussed above are compatible with any dosage form or route of administration. However, bile acids and other TGR5 agonists will most typically be used orally in the form of a tablet, capsule or powder and these forms are most preferred. All dosage forms may be prepared using methods that are standard in the art (see e.g., Remington's Pharmaceutical Sciences, 16th ed. A. Oslo. ed., Easton, Pa. (1980)).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

C. Dosages

Dosages will be determined using methods well established in clinical medicine. General guidance concerning appropriate amounts of bile acids is available based upon methods that already exist in the art and from the studies described in the Examples section below. In general, it is expected that a human will take between 0.1 mg/kg body weight/day and 25 mg/kg/day and preferably between 0.5 and 20 mg/kg/day and comparable molar amounts of other TGR5 agonists. However, these are simply guidelines and the actual dose may be adjusted based upon factors unique to each individual. Daily dosages may be provided in either a single or multiple regimen. A single type of bile acid or other TGR5 agonist may be taken or more than one type may be taken. In addition, these compounds may be combined with other nutritional supplements or medications.

D. Assays for Identifying Compounds With Therapeutic Potential

The invention includes assay methods that can be used to screen for compounds that are likely to be useful in treating the diseases and conditions described above. One way to accomplish this is to determine whether a compound increases the activity of D2. Any cell that expresses D2 can be used for this purpose but human skeletal muscle cells or cells derived from mouse brown adipose tissue are preferred. The compound being tested is incubated with the cells for a set time period and a determination of D2 is then made using procedures well known in the art. Assays may be based upon either the amount of D2 present (e.g., determined using PCR as discussed in the Examples section or by the amount of protein present using antibody specific for D2) or the amount of D2 activity present (e.g., using an enzyme assay). The results obtained are compared with those from control incubations carried out in a similar manner but in the absence of the test compound. Compounds that cause an increase in D2 relative to controls have potential as therapeutic agents.

Agonists of TGR5 other than bile acids should also be effective therapeutic agents. Thus, screening assays may be directed at determining whether a test compound binds to and activates TGR5. These assays will involve main two steps: a) determining if there is specific binding; and b) determining if a compound that binds specifically is activating the receptor. Receptor binding assays suitable for use in connection with TGR5 are well known in the art and may take several different forms (see e.g., Chard, T., “An Introduction to Radioimmune Assay and Related Techniques,” in Laboratory Techniques in Biochemistry and Molecular Biology, North Holland Publishing Co., N.Y. (1978)). The essential feature of these assays is that a source of receptor (TGR5) is incubated together with a ligand known to bind to the receptor (e.g., cholic acid) and with the compound being tested for binding activity.

The preferred source for TGR5 is cells, preferably skeletal muscle cells, or cells recombinantly transformed to express the receptor. In order to make recombinant cells, an appropriate expression vector coding for TGR5 must be constructed. The complete amino acid (and gene) sequences of TGR5 from several species have been reported in the art (see Kawamata, et al, J. Biol. Chem. 278:9435-9440 (2003); Maruyama, et al. Biochem. Biophys. Res. Commun. 298:714 (2002)) and may be used in these vectors. The most preferred TGR5 sequence is from the human (GenBank accession number AB089307). Other, less preferred sequences that could be used include the sequence from the mouse (GenBank accession no. AB89308, see also EMBL accession no. Q805556); rabbit (GenBank accession no. AB089308); cow (GenBank accession no. AB089306); and rat (GenBank accession no. AB089310). (All GenBank references hereby being incorporated herein by reference.) Standard techniques for constructing vectors and transforming host cells may be used (see e.g., Sambrook et al., Molecular Cloning: A Laboratory Manual, 2nd ed. Cold Spring Harbor (1989)). When cell isolates or cells lines are used in assays, they should be selected based upon their having a substantial amount of TGR5.

Binding assays may be performed either with intact cells or with membranes prepared from the cells (see e.g., Wang, et al., Proc. Natl. Acad. Sci. U.S.A. 90:10230-10234 (1993)). The cells (or membranes) are incubated with a ligand specific for TGR5 and with a preparation of the compound being tested. After binding is complete, receptor is separated from the solution containing ligand and test compound, e.g., by filtration, and the amount of binding that has occurred is determined. Preferably, the ligand used is a bile acid detectably labeled with a radioisotope such as ¹²⁵I. However, if desired, fluorescent or chemiluminescent labels can be used instead. Among the most commonly used fluorescent labeling compounds are fluorescein isothiocynate, rhodamine, phycoerythrin, phycocyanin, allophycocyanin, o-phthaldehyde and fluorescamine. Useful chemiluminescent compounds include luminol, isoluminol, theromatic acridinium ester, imidazole, acridinium salt, and oxalate ester.

Nonspecific binding may be determined by carrying out the binding reaction in the presence of a large excess of unlabelled ligand. For example, labeled bile acid may be incubated with receptor and test compound in the presence of a thousand-fold excess of unlabelled bile acid. Nonspecific binding should be subtracted from total binding, i.e. binding in the absence of unlabeled ligand, to arrive at the specific binding for each sample tested. Other steps such as washing, stirring, shaking, filtering and the like may be included in the assay as necessary. Typically, wash steps are included after the separation of membrane-bound ligand from ligand remaining in solution and prior to quantitation of the amount of ligand bound, e.g., by counting radioactive isotope. The specific binding obtained in the presence of test compound is compared with that obtained in the presence of labeled ligand alone to determine the extent to which the test compound has displaced bile acid.

In performing binding assays, care must be taken to avoid artifacts which may make it appear that a test compound is interacting with the TGR5 receptor when, in fact, binding of labeled ligand is being inhibited by some other mechanism. For example, the compound being tested should be in a buffer which does not itself substantially inhibit the binding of bile acid to TGR5 and should, preferably, be tested at several different concentrations. Preparations of test compound should also be examined for proteolytic activity and it is desirable that antiproteases be included in assays. Finally, it is highly desirable that compounds identified as displacing the binding of ligand to TGR5 be reexamined in a concentration range sufficient to perform a Scatchard analysis on the results. This type of analysis is well known in the art and can be used for determining the affinity of a test compounds for receptor (see e.g., Ausubel, et al., Current Protocols in Molecular Biology, 11.2.1-11.2.19 (1993); Laboratory Techniques and Biochemistry and Molecular Biology, Work, et al., ed., N.Y. (1978)). Computer programs may be used to help in the analysis of results (see e.g., Munson, P., Methods Enzymol. 92:543-577 (1983); McPherson, G. A., Kinetic, EBDA Ligand, Lowry-A Collection of Radioligand Binding Analysis Programs, Elsevier-Biosoft, U.K. (1985)).

Once it has been determined that a compound binds with specificity to TGR5, the next step is to determine whether binding is accompanied by receptor activation. This can be accomplished using any cellular activity that TGR5 is known to induce. For example, the mRNA levels of a gene known to be induced by TGR5 may be determined both before and after binding. Preferably, however, activation will be assessed based upon whether binding results in an increase in intracellular adenyl cyclase activity. Many methods for determining cAMP levels and adenyl cyclase activity have been described in the art and are suitable for use in connection with the present invention. For example, the Biotrack cAMP Enzyme-immunoassay System (Amersham) or the Cyclic AMP [3H] Assay System (Amersham) are commercially available assays that could be used. Typically, adenyl cyclase assays will be performed separately from binding assays, but it may also be possible to perform binding and adenyl cyclase assays on a single preparation of cells. Compounds that both bind to TGR5 with specificity and that then increase adenyl cyclase have the greatest potential as therapeutics.

### EXAMPLES

### Example 1

### Bile Acids Lower Triglyceride Levels Via a Pathway Involving FXR, SHP and SREBP-1c

The present example describes the effects of bile acids on triglyceride (TG) homeostasis using a combination of molecular, cellular, and animal models. Cholic acid (CA) prevents hepatic TG accumulation, VLDL secretion, and elevated serum TG in mouse models of hypertriglyceridemia. At the molecular level, CA decreases hepatic expression of SREBP-1c and its lipogenic target genes. Through the use of mouse mutants for the short heterodimer partner (SHP) and liver X receptor (LXR) α and β, we demonstrate the critical dependence of the reduction of SREBP-1c expression by either natural or synthetic farnesoid X receptor (FXR) agonists on both SHP and LXRα and LXRβ. These results suggest that strategies aimed at increasing FXR activity and the repressive effects of SHP may be used to correct hypertriglyceridemia.

A. Introduction

Hypertriglyceridemia is a strong predictor of coronary heart disease. This is mainly attributed to the inverse relationship between serum triglycerides (TGs) and HDL cholesterol, since low levels of HDL increase the risk of vascular disease. However, several recent findings have provided compelling evidence that TGs are also an independent risk factor (Cullen, P., Am. J. Cardiol. 86:943-949 (2000); Ginsberg, H. N., Am. J. Cardiol. 87:1174-1180 (2001)). In most cases, hypertriglyceridemia is secondary to a westernized life-style, which is characterized by a lack of physical exercise and increased caloric intake. Such a life-style is associated with visceral fat accumulation, insulin resistance, and hypertriglyceridemia (type IIb and type IV hyperlipoproteinemia), as a consequence of an increased production of VLDL by the liver.

Bile acids have long been known to affect TG homeostasis. In humans, bile acid-binding resins induce the production of VLDL TGs (Angelin, et al., J. Lipid Res. 19:1017-1024 (1978); Beil, et al., Metabolism. 31:438-444 (1982); Crouse, J. R., Am. J. Med. 83:243-248 (1987)), whereas treatment of cholesterol gallstones with the bile acid chenodeoxycholic acid (CDCA) has been shown to reduce hypertriglyceridemia (Angelin, et al., J. Lipid Res. 19:1017-1024 (1978); Bateson, et al., Br. J. Clin. Pharmacol. 5:249-254 (1978); Carulli, et al., J. Clin. Pharmacol. 21:436-442 (1981)). The mechanism underlying this reciprocal relationship between bile acid biosynthesis and TG production has remained elusive, but two possible explanations have been postulated. At the transcriptional level, bile acids, which are the endogenous ligands of the farnesoid X receptor (FXR, NR1H4), activate the transcription of several genes that could modulate TG levels, such as the atypical nuclear receptor short heterodimer partner (SHP, NR0B2) (Goodwin, et al., Mol. Cell. 6:517-526 (2000); Lu, et al., Mol. Cell. 6:507-515 (2000); PPARα (Pineda Torra, et al., Mol. Endocrinol. 17:259-272 (2003)), and ApoC-II (Kast, et al., Mol. Endocrinol. 15:1720-1728 (2001)). Alternatively, at the metabolite level, a reduction in bile acid biosynthesis could increase hepatic cholesterol and oxysterol levels, which will influence the function of the lipogenic SREBP-1c by attenuating its processing and activation. This could lead to decreased TG production (Beigneux, et al., J. Clin. Invest. 110:29-31 (2002); Pullinger, et al. J. Clin. Invest. 110:109-117 (2002)). Here, we provide evidence in support of the first hypothesis and show that bile acids, by activating FXR, induce the expression of SHP. SHP then interferes with SREBP-1c expression by inhibiting the activity of LXR and eventually other transcription factors that stimulate SREBP-1c expression.

B. Material and Methods

Materials

Cholesterol, 22(R)-hydroxycholesterol, 25-hydroxycholesterol, CA, and CDCA were obtained from Sigma-Aldrich (St. Quentin Fallavier, France).

Plasmids

pCMX-SHP was obtained by insertion of a PCR product corresponding to the mouse SHP cDNA into the pCMX vector. pCMX-liver receptor homolog-1 (pCMX-LRH-1) was produced by insertion of a PCR product corresponding to the mouse LRH-1 cDNA into pCMX. The pCMX-LXRα expression vector was as described(Repa, et al., Genes Dev. 14:2819-2830 (2000)); the pSG5-retinoid X receptor α (pSG5-RXRα) expression vector was a gift of P. Chambon (Institut Clinique de la Souris, Illkirch, France). The SREBP-1c promoter luciferase reporter plasmids were generated by PCR amplification of promoter fragments corresponding to sequences located between −1070 to −51 of the mouse SREBP-1c gene. The PCR product was ligated into the pGL3 basic vector (Promega, Madison, Wis., USA). The reverse primer used for each promoter construct is 5′-CTTCCGCGCCGATTTCACCTG-3′ (SEQ ID NO:1). The different forward primers used for each construct are as follows: pSREBP-1c1070-Luc (5′-ACCCCTCAGACTGTGTGAGT-3′) (SEQ ID NO:2); pSREBP-1c571-Luc (5′-CTA GCTAGATGACCCTGCACCACCAA-3′) (SEQ ID NO:3); pSREBP-1c327-Luc (5′-TTGCCTGT GCGGCAGGGGTTGGGACGA-3′) (SEQ ID NO:4); pSREBP-1c276-Luc (5′-CGCGCTGGCGCAGACGCGGTTAAA-3′) (SEQ ID NO:5); and pSREBP-1c151-Luc (5′-CTGCTGATTGGCCATGTGCGCTCA-3′) (SEQ ID NO:6). The primers were tailed with either a KpnI site (forward) or a BglII site (reverse). The LXR response elements in pSREBP-1c324-Luc were mutated for promoter analysis using the Quick Change Site-Directed Mutagenesis Kit (Stratagene, La Jolla, Calif., USA). All constructs were verified by sequence analysis.

Animals

Male C57BL/6J, KK-Ay, and ob/ob (C57BL/6J background) mice, 6-10 weeks of age, were obtained from Elevage Janvier (Le Genest St. Isle, France), CLEA Japan Inc. (Tokyo, Japan), and The Jackson Laboratory (Bar Harbor, Me., USA), respectively. All mice were maintained in a temperature-controlled (23° C.) facility with a 12-hour light/dark cycle and were given free access to food and water. The study protocols were approved by the institutional review boards. The body weight and food intake were measured every second day. The control and high-fat diet was obtained from UAR (Villemoisson sur Orge, France). The control diet (EQ12310) contained 16.8% protein, 73.5% carbohydrate, and 4.8% fat, whereas the high-fat diet (EQ/ID12309) contained 23.0% protein, 35.5% carbohydrate, and 35.9% fat. For treatment with bile acids, mice were fed diets with 0.5% (w/w) CA. The mice were fasted 4 hours before harvesting blood for subsequent lipid measurements, and tissues for RNA isolation, lipid measurements, and histology. Male SHP−/−mice and littermate SHP+/+ controls (C57/BL6-A129/SvJ mixed strain) of 8 weeks were fed the CA diet as just described or gavaged with GW4064 dissolved in corn oil at 30 mg/kg. Male wild-type and LAR(4-1-mice (C57/BL6-A129/SvJ mixed strain; Repa, et al., Genes Dev. 14:2819-2830 (2000)) 10-14 weeks old were fed a powdered chow diet (Harlan Teklad 7001, Madison, Wis., USA) and, where indicated, mixed with 0.5% CA. A T0901317 suspension prepared in a 1% carboxymethylcellulose solution was used for gavage at 30 mg/kg. KK-Ay and ob/ob mice on the control diet were treated daily by intraperitoneal injection for 1 week with GW4064 prepared in a 1% carboxymethylcellulose solution at 10 mg/kg.

Lipid Measurements and Liver Function Tests

Serum total cholesterol, TGs, and lipoprotein cholesterol and TG profiles were measured as described (Rocchi, et al., Mol Cell. 8:737-747 (2001); Picard, et al., Cell 111:931-941 (2002)). For measurement of liver TGs and cholesterol content, a liver was homogenized in chloroform/methanol (2:1 v/v) using a Polytron tissue grinder (Kinematica AG, Luzern, Switzerland). Lipid extracts were prepared by the classical Folch method. Extracts were dried under N₂ flow and resuspended in isopropanol. For in vivo measurements of VLDL TG production, mice were injected with Tyloxapol (Sigma-Aldrich) (500 μg/g body weight), after which blood samples were taken at various time points (Boisfer, et al., J. Biol. Chem. 274:11564-11572 (1999)).

Cell Culture, Transient Transfection, and Luciferase Assays

The rat hepatoma cell line McA-RH7777 was obtained from ATCC (Manassas, Va., USA). Cells were grown at 37° C. in a humidified atmosphere of 5% CO₂/95% air. Transfections were performed in 96-well plates using Lipofectamine 2000 (Invitrogen, Cergy Pontoise, France). Each well contained 64 ng luciferase reporter and 4 ng of β-galactosidase expression plasmid. We transfected 16 ng RXRα, 16 ng LXRα, 16 ng LRH-1, 32 ng SHP expression plasmids, or the corresponding empty expression vectors. After 6 hours of incubation with the DNA-lipofectamine complexes, the transfection medium was exchanged for medium with or without the indicated ligands. Ligands for LXR and RXR were dissolved in ethanol or DMSO and added to the cells in DMEM supplemented with 10% lipoprotein-deficient serum. To suppress SREBP-1c activation by processing, leading to feed-forward induction of SREBP-1c expression, the medium used for the SREBP-1c promoter study was also supplemented with 10 μg/ml cholesterol and 1 μg/ml 25-hydroxycholesterol. Luciferase measurements were normalized to β-galactosidase activity.

Hepatocytes

Hepatocytes were isolated from wild-type male mice 8-10 weeks old. Animals were anesthetized with CO₂ and livers were perfused with 40 ml Liver Perfusion Medium (Invitrogen) followed by 30 ml Liver Digestion Medium (Invitrogen), both at a flow rate of 5 ml/min. Isolated hepatocytes were resuspended in DMEM supplemented with 20% (v/v) FCS, 100 units/ml penicillin, and 100 units/ml streptomycin, and placed in plates coated with collagen IV at a density of 1×10⁵ cells/cm². After a 4-hour incubation at 37° C. in an atmosphere of 5% CO₂, the medium was changed to DMEM supplemented with 10% (v/v) FCS, 100 units/ml penicillin, 100 units/ml streptomycin, 0.5 μM dexamethasone (Sigma-Aldrich), and 0.4 μM insulin (Sigma-Aldrich). After 16 hours, the medium was changed into medium supplemented with CDCA and ligands for LXR and RXR. After an additional 18-hour incubation, cells were harvested for RNA isolation.

Expression-level Analysis

Total RNA was extracted from frozen tissue samples or cells using the RNeasy kit (QIAGEN, Courtaboeuf, France). cDNA was synthesized from total RNA with the SuperScript First-Strand Synthesis System (Invitrogen) and random hexamer primers. The real-time PCR measurement of individual cDNAs was performed using SYBR green dye to measure duplex DNA formation with the LightCycler System (Roche Diagnostics, Meylan, France). The sequences of the primer sets used are available online at http://www-igbmc.ustrasbg.fr/Departments/Dep_V/Dep_VA/Publi/Paper.html.

Statistical Analysis

Values were reported as mean ±SE. Statistical differences were determined by either a Student's τ test or an ANOVA followed by a Bonferroni or Dunnett post test. Statistical significance is displayed as * (P <0.05) or ** (P <0.01).

C. Results

Cholic Acid Lowers Serum TGs in KK-Ay Mice

To study the TG-lowering effect of bile acids in vivo, we first explored the effect of feeding CA on TG homeostasis in KK-Ay mice. KK is an inbred strain that develops type 2 diabetes mellitus with only mild obesity, even after maturity. Introduction of the lethal yellow mutation in the agouti gene (Ay) causes overt type 2 diabetes and massive obesity with a relatively late onset. These KK-Ay mice are characterized by a severe prolonged hyperinsulinemia, hyperglycemia, and hyperlipidemia. Under basal conditions the elevation of TGs is only moderate (192 ±16 mg/dl), but can be further increased by a high-fat diet (273 ±14 mg/dl). This elevation of serum TG levels on a high-fat diet is not observed in normal C57BL/6J mice, making the KK-Ay mice a unique model to study diet-induced hypertriglyceridemia. In addition, the elevation of serum TG is progressive and dependent on the age of the KK-Ay mice. At 7 weeks of age, the hypertriglyceridemia is moderate (192 ±16 mg/dl, whereas at 12 weeks of age, it is severe (453 ±24 mg/dl).

Male KK-Ay mice were given normal chow or high-fat diets with or without 0.5% CA for 1 week. The CA-containing diets were well tolerated, and food intake was not affected during a 1-week and a 3-week study. Interestingly, CA robustly lowered circulating TG levels in both KK-Ay mice on a normal-chow (51%) and on a high-fat diet (65%). Total plasma cholesterol was also decreased (24% on a normal-chow diet and 35% on a high-fat diet). CA feeding decreased plasma FFAs, particularly in mice on the high-fat diet. High-performance liquid chromatography analysis of lipoproteins demonstrated that CA feeding decreased plasma TGs primarily by decreasing VLDL TGs, and decreased cholesterol primarily by decreasing HDL cholesterol. CA feeding increased LDL cholesterol levels.

To determine whether the observed effects of CA were mediated through FXR, we treated chow-fed KK-Ay and ob/ob mice during 1 week with the synthetic FXR agonist GW4064. GW4064 potently lowered serum TG levels in both the KK-Ay and ob/ob mice, suggesting that the observed effects are mediated by FXR. In addition, this experiment suggests that the TG-lowering effect of FXR activation is not restricted to KK-Ay mice, a model for diet-induced hypertriglyceridemia, but also occurs in another model of hypertriglyceridemia, the ob/ob mouse.

CA lowers hepatic TG levels

Administration of CA to KK-Ay mice for 3 weeks also changed liver morphology. Livers of chow-fed KK-Ay mice have a pale color, suggestive of increased lipid storage. Livers of animals that were treated with CA were less pale and had a more normal reddish appearance. This effect of bile acids on hepatosteatosis was more pronounced in mice fed high-fat diets, which showed an impressive return to a normal morphology. H&E-stained sections of the livers of animals on the high-fat diet with CA showed much lower levels of unstained inclusions. Staining of these liver sections with Oil Red 0 (Sigma-Aldrich) demonstrated that CA-treated animals accumulated less neutral lipids, an effect consistent with both gross morphological appearance and H&E staining. Consistent with the morphological appearance, livers of CA-treated animals contained significantly lower amounts of TGs in animals on chow or high-fat diet. In contrast, CA feeding increased hepatic cholesterol, particularly in the animals fed high-fat diets.

To assess whether the decrease in liver TG content is associated with decreased export, we measured VLDL production in KK-Ay mice after 1 week of CA feeding. CA feeding significantly decreased liver VLDL production on both a chow and high-fat diet. In a similar experiment, a 1-week administration of GW4064 also significantly lowered VLDL secretion in KK-Ay mice, again suggesting that this effect is mediated via FXR.

CA Decreases Expression of SREBP-1c and Other Lipogenic Genes

To better understand the molecular mechanism underlying the TG-lowering effect of bile acids, we used quantitative RT-PCR to measure hepatic mRNA levels of several important proteins involved in lipid homeostasis in KK-Ay and C57BL/6J mice after 1 and 7 days of CA supplementation of a chow diet. No major changes were observed in the expression of several transcription factors involved in liver lipid and bile acid homeostasis, such as LXRA, LRH-1 (NR5A2), FXR, and SREBP-2. Expression of the LDL receptor (LDL-R) and genes involved in cholesterol biosynthesis had a tendency to decrease, whereas genes involved in encoding cholesterol transport proteins (ABCA1 and ABCG5) were slightly increased, although these differences never reached statistical significance. Expression of genes encoding enzymes involved in fatty acid and TG biosynthesis, such as AceCS, ME, and SCD-1, was significantly reduced by CA feeding. This reduction is strongest after 1 day of treatment and is mitigated somewhat after 7 days of treatment.

To verify whether an increase in fatty acid, β-oxidation contributed to the TG-lowering effects in our studies, we measured the hepatic expression levels of liver carnitine palmitoyltransferase I (CPT-I), medium-chain acyl-CoA dehydrogenase (MCAD), and long-chain acyl-CoA dehydrogenase (LCAD). In C57BL/6J mice, the expression of MCAD and LCAD was significantly decreased after 1 and 7 days of treatment with CA. Interestingly, in KK-Ay mice there was no change in the expression of these genes. Thus, increased expression of genes involved in β-oxidation of fatty acids is not responsible for the decrease in serum TG. Aside from these differences in genes involved in β-oxidation, no major differences were observed between the results obtained in KK-Ay and C57BL/6J mice.

Interestingly, the changes in expression of genes involved in lipogenesis were paralleled by changes in SREBP-1c expression. Furthermore, expression of SHP showed an opposite pattern, with a robust induction upon treatment with CA. SHP is an FXR target gene that represses the activity of several nuclear receptors, including LRH-1 and LXR, which are essential for the transcription of cholesterol 7α-hydroxylase (CYP7A1), the rate-limiting enzyme in bile acid biosynthesis. This FXR-mediated SHP induction underlies the negative-feedback regulation of bile acid biosynthesis. The effect of CA on lipogenesis seems to be specific for the liver, since in white adipose tissue, none of the lipogenic genes were lowered in expression in response to CA treatment.

The activity of the SREBP-1c promoter is attenuated by bile acids and SHP

We examined the ability of CDCA to lower expression of endogenous SREBP-1c and its target genes in vitro in mouse primary hepatocytes. The SREBP-1c promoter had previously been shown to be regulated by LXR, an effect that contributes to the TG-raising activity of LXR agonists. We confirmed the induction of endogenous SREBP-1c expression by RXR and LXR ligands and showed that this expression decreased dose-dependently by increasing amounts of CDCA. The expression of the lipogenic target genes of SREBP-1c decreased likewise. This reduction was most robust for SCD-1, but expression of AceCS and ME was also reduced. SHP expression was increased by the addition of CDCA. These results demonstrate that, both in vivo and in vitro, the expression of endogenous SREBP-1c, as well as the lipogenic enzymes that are regulated by SREBP-1c, is significantly affected by bile acids.

To examine this potential role of bile acids in the regulation of SREBP-1c expression, we cloned the mouse SREBP-1c promoter and generated a luciferase reporter construct. McA-RH7777 rat hepatoma cells were cotransfected with this construct and a LXRα expression vector and treated with LXR and RXR agonists in the presence or absence of CA or CDCA. SREBP-1c promoter activity was induced by transfection of LXRα and/or the addition of its ligands. This increase was attenuated when cells were incubated with either of the bile acids. Furthermore, induction of SREBP-1c promoter activity by LXR and RXR agonists does not depend on the overexpression of LXR, showing that McA-RH7777 cells have endogenous LXR activity.

The effect of CA on the expression of lipogenic genes, as well as the opposite changes in gene expression of SREBP-1c and SHP, prompted us to analyze the contribution of the FXR-SHP cascade to the regulation of SREBP-1c gene expression. We therefore generated a number of reporter constructs containing 5′ nested deletions or point mutations in the two previously characterized LXR response elements (LXRREs). As expected, SREBP-1c promoter activity is induced by overexpression of the RXR/LXRα heterodimer in rat hepatoma McA-RH7777 cells in the presence of a natural LXR agonist and a synthetic RXR ligand. When the LXRRE sites are mutated or deleted, basal activity of the promoter is markedly reduced. It is probable that this effect is conserved between mice and humans, since SREBP-1c expression is also induced by an LXR agonist in human hepatoma cells and primary hepatocytes. In addition, the two LXRREs are highly conserved between the mouse and the human SREBP-1c promoter. Transfection of LRH-1 induced SREBP-1c promoter activity and improved the magnitude of induction by ligand-activated LXR, suggesting that LRH-1 acts as a competence factor for LXR as was reported for a number of genes. The induction of the SREBP-1c promoter by LRH-1 was lost after deletion of LXRREa. The finding that the promoter region between −327 and −276 contained no consensus LRH-1 response element (LRH-1RE) suggested that the induction might be mediated via LXRREa.

Mutation of the 5′ extension of this LXRRE, which does not disrupt the LXRRE consensus sequence itself, did lead to the loss of response to LRH-1 which is in line with the suggestion that the LXRREa might mediate the response of SREBP-1c to LRH-1. Interestingly, cotransfection with SHP potently attenuated the induction of the SREBP-1c reporter in the presence of LRH-1, LXR, and RXR. From this we conclude that SHP regulates the SREBP-1c promoter. Due to technical problems (the loss of basal promoter activity after the mutagenesis of the LXRREs), we cannot attribute this effect with certainty to a particular site in the promoter.

CA attenuates LXR agonist-induced lipogenesis in vivo

To study the inhibition of the SREBP-1c promoter by CA in more detail, we fed C57BL/6J mice chow or chow supplemented with 0.5% CA for 1 day. Mice were gavaged with the LXR agonist T0901317 or vehicle, and the different diets were continued for one more day, after which the mice were sacrificed. We confirmed the previously observed induction of liver weight and serum TGs by LXR agonists on a chow diet. Coadministration of CA completely prevented these effects. As expected, both LXR (SREBP-1c, CYP7A1, ABCA1, ABCG5, ABCG8, and ANGPTL3) and SREBP-1c (ME, ACC1, and ACC2) target genes were induced by administration of the LXR ligand. CA coadministration increased SHP expression and prevented the induction of SREBP-1c and its target genes. Interestingly, some LXR target genes were downregulated by CA (SREBP-1c, CYP7A1, and ANGPTL3), whereas others (ABCA1, ABCG5, and ABCG8) were not. This led us to conclude that not all LXR target genes are responsive to inhibition by SHP, suggesting that besides LXR there might be additional factor(s) targeted by CA to explain their efficient downregulation.

Attenuation of the TG-lowering effects of FXR agonists in SHP−/− mice

To critically test the role of SHP in the lowering of TG biosynthesis, we administered either a diet containing 0.5% CA or the synthetic FXR agonist GW4064 to wild-type and SHP-null mice and measured serum TGs after 0, 3, and 7 days. CA and GW4064 significantly lowered serum TGs in the SHP+/+ mice. CA seemed more potent in decreasing serum TGs, an effect that may be attributed to the poor pharmacokinetic profile of GW4064. More importantly, this decrease in serum TGs was completely abolished in the SHP−/− mice. To determine whether this attenuation of the TG-lowering effects of FXR agonists in SHP−/− mice is paralleled by similar changes at the molecular level, we measured the hepatic expression of SREBP-1c, ME, CYP7A1, and ANGPTL3 in animals sacrificed after a 1-day treatment with the different FXR agonists. Expression of all four genes was significantly reduced in the SHP+/+ mice that received CA or GW4064. This is in sharp contrast with the results obtained in the SHP−/− mice, in which no decrease in this expression was detected. The attenuation of CYP7A1 downregulation in SHP−/− mice upon FXR agonist administration is in apparent contrast with earlier studies. This is probably a reflection of the differences in experimental approach (1 day vs. 7 days and 0.5% CA vs. 1% CA treatment) that favored activation of secondary regulatory pathways in the earlier studies (Wang, et al., Dev. Cell. 2:721-731 (2002)). These data further support that SHP plays an essential role in the SREBP-1c-mediated downregulation of lipogenesis in mice treated with bile acids.

LXR is essential for SHP-mediated lowering of TGs

To determine whether LXR is essential for the TG-lowering effect of bile acids, we used LXRα/LXRβ double-knockout mice (LXRα^{-/-}). Wild-type and LXRα/β^{-/-} mice were fed with a diet containing 0.5% CA for 3 days, after which we measured serum TGs and hepatic gene expression of SREBP-1c, ME, CYP7A1, and SHP. As a result of the loss of LXR transactivation in the LXRα/β^{-/-} mice, the basal level of expression of the LXR (SREBP-1c and CYP7A1) and SREBP-1c (ME) target genes is significantly reduced, causing a lower basal serum TG level. In wild-type animals, CA treatment decreased serum TG subsequent to a decrease in the expression SREBP-1c and ME. The decrease in serum TGs, SREBP-1c, and ME is not observed in the LXRα/β^{-/-} mice. Though less pronounced in LXRα/β^{-/-} mice, SHP was induced significantly in both wild-type and LXRα/β^{-/-} animals. These experiments demonstrate that LXR is essential for the SHP-mediated lowering of TGs in vivo.

D. Discussion

We here investigated the effects of bile acids on TG homeostasis and confirmed that bile acids have a beneficial effect in hypertriglyceridemia. For these studies, we used KK-Ay mice, a model for obesity and type 2 diabetes mellitus. On a normal diet these mice have moderately elevated serum TGs, which increase further upon high-fat feeding. This makes KK-Ay mice the first mouse model for diet-induced type IIb and type IV hyperlipoproteinemia. Administration of CA to KK-Ay mice reduced serum and hepatic TG concentrations and VLDL secretion in mice fed either a chow or a high-fat diet. The TG-lowering effect was also present in ob/ob mice, another model of obesity, insulin resistance, and hypertriglyceridemia. These results are consistent with earlier studies indicating a reciprocal relationship between bile acid pool and TG production. For example, bile acid-binding resins have been reported to induce the production of VLDL TGs. In fact, increased TG levels are often an undesirable side effect of the use of these resins to manage lipid disorders. In addition, elevated TGs have recently been reported in patients with CYP7A1 deficiency (Pullinger, et al., J. Clin. Invest. 110:109-117 (2002)). Whereas mice with a targeted disruption of CYP7A1 do not have a manifest hypertriglyceridemia (Schwarz, et al., J. Biol. Chem. 271:18024-18031 (1996), CYP27-knockout mice, which lack another gene participating in the conversion of cholesterol to bile acids, do have hypertriglyceridemia (Repa, et al., J. Biol. Chem. 275:39685-39692 (2000)). Interestingly, this elevation of TGs could be attenuated by the addition of bile acids in the diet of the CYP27-knockout mice. Furthermore, when compared with normal subjects, hypertriglyceridemic patients have a decreased level of the ileal sodium bile acid transporter, resulting in an impaired enterohepatic recycling of bile acids (Duane, et. al, J. Lipid Res. 41:1384-1389 (2000)). Finally, addition of bile acids to cultured rat and human hepatocytes decreased VLDL secretion (del Pozo, et al., Biol. Chem. Hoppe-Seyler. 368:887-893 (1987); Lin, et al., Biochem. J. 316:531-538 (1996); Lin, et al., Hepatology. 23:218-228 (1996)), underscoring the crucial role of the liver in this process.

Besides lowering serum TGs, CA also lowered serum HDL cholesterol levels in our study. These results are consistent with other recent reports in rodents and humans in which bile acids influence expression of cholesteryl ester transfer protein, phospholipid transfer protein, and ApoA-I (Luo, et al., J. Biol. Chem. 276:24767-24773 (2001), Srivastava, et al., Eur. J. Biochem. 267:4272-4280 (2000); Urizar, et al, J. Biol. Chem. 275:39313-39317 (2000); Claudel, et al., J. Clin. Invest. 109:961-971 (2002)). More importantly, the inverse relationship between HDL and TGs, often troublesome in determining whether TGs are an independent risk factor for coronary heart disease, seems uncoupled by CA.

The molecular mechanism for the TG-lowering effects of bile acids has remained elusive. We show here that bile acids lower serum TG levels by targeting SREBP-1c gene expression. Since SREBP-1c is the main regulator of hepatic fatty acid and TG biosynthesis, reducing its expression would be expected to decrease both hepatic TG storage and VLDL production, as we have shown in KK-Ay mice. We confirmed here that the basal transcription level of SREBP-1c critically depends on LXR. We furthermore demonstrate that activation of FXR, by natural and synthetic agonists, increases SHP levels, which in turn reduces SREBP-1c expression. This is in perfect analogy to the mechanism proposed to explain the reduction of CYP7A1 expression by bile acids, which also invoked SHP as a mediator. We validated the central role of SHP in the process of inhibiting SREBP-1c using SHP−/− mice. In these mice, TG levels are not lowered in response to natural (bile acids) and synthetic (GW4064) FXR agonists, which is paralleled at the molecular level by an attenuated response of SREBP-1c and its target gene ME.

The combined use of synthetic FXR agonists and SHP−/− mice rules against the possibility that bile acid-activated, but FXR- and SHP-independent, cell signaling pathways are important mediators in the TG-lowering effect. An important role of LXR as a key player in the regulation of SREBP-1c expression by CA was provided by the lack of a TG-lowering effect subsequent to FXR activation in LXRα/β^{-/-} mice. The result of the experiment in which we coadministered LXR and FXR agonists, however, showed that the expression of some LXR target genes is decreased in response to CA (SREBP-1c, CYP7A1, and ANGPTL3), while the expression of others is not (ABCA1, ABCG5, and ABCG8), suggesting that besides LXR there is an additional factor targeted by CA to mediate downregulation of these genes. LRH-1 could be that additional factor in view of its role as a competence factor for LXR gene regulation. We were unable, however, to unequivocally confirm or rebut the hypothesis that LRH-1 plays a role in this mechanism because of a total absence of SREBP-1c promoter activity in vitro after mutation of the LXRREs, the absence of a consensus LRH-1RE, and the lack of a mutant LRH-1 mouse model.

The response of SREBP-1c and its target genes to bile acids is strongest after 1 day of treatment and seems to be attenuated after 7 days of treatment. The TG-lowering effects of CA in the KK-Ay mice, however, were maintained for up to 3 weeks. We believe that SREBP-1c is the initial major target of bile acids to decrease TG levels. The persistence of TG lowering after chronic elevation of the bile acid pool suggests that bile acids could have additional effects on TG homeostasis. Other possible explanations have been put forward that are not mutually exclusive. In cultured human hepatocytes, bile acids induce the expression of PPARα and its target genes involved in fatty acid β-oxidation, such as CPT-I. We found that CA feeding decreased mRNA levels of genes involved in β-oxidation in C57BL/6J mice, consistent with the previously reported inhibition of PPARα by bile acids (Sinal, et al., J. Biol. Chem. 276:47154-47162 (2001)). Expression of β-oxidation genes, furthermore, did not change in KK-Ay mice, ruling against a contribution of changes in fatty acid β-oxidation to explain the decrease in serum TG levels after CA feeding. ApoC-II, an activator of lipoprotein lipase, an enzyme that hydrolyzes TGs from VLDL and chylomicrons and as such could lower serum TG levels, has been reported to be an FXR target gene. In addition, we show here that, similar to SREBP-1c, the expression of the LXR target gene ANGPTL3 is lowered by CA feeding via a SHP-dependent mechanism. ANGPTL3 can increase serum TGs by inactivating lipoprotein lipase. Thus besides reducing hepatic VLDL production via effects on SREBP-1c, bile acids will also increase VLDL clearance via effects on ApoC-II and ANGPTL3. Very recently, Zhang et al. reported that FXR mRNA levels are increased by PPARγ and HNF4α. These investigators further show that PGC-1α is a coactivator for FXR. Fasting markedly induces hepatic PGC-1α levels. Under this condition, PGC-1α and FXR cooperate to maintain energy homeostasis by decreasing serum TG levels via effects on VLDL clearance.

Since most mouse strains are very resistant to food-induced hyperlipoproteinemia, expression of the active form of SREBP-1c in the liver did not increase serum TGs despite increased hepatic TG synthesis. Several other lines of evidence, however, point toward an important role for liver SREBP-1c in lipogenesis, obesity, and hyperlipidemia. Hepatic SREBP-1c gene expression is increased in animal models of obesity and type 2 diabetes. In addition, obese patients have an increased hepatic lipogenesis (and often a subsequent hypertriglyceridemia) that could contribute to hepatic insulin resistance and their excessive fat mass, which decreased after energy restriction (Diraison, et al., Am. J. Physiol. Endocrinol. Metab. 282:E46-E51 (2002)).

The FXR-SHP-SREBP-1c regulatory cascade that we propose can explain a number of previous results linking FXR activity to TG levels in liver and serum. FXR−/− mice show decreased basal expression of SHP and have increased serum and hepatic TG levels (Sinal, et al., Cell 102:731-744 (2000)). Moreover, plasma TG decreased when rats were treated for 7 days with the synthetic FXR ligand GW4046 (Maloney, et al., J. Med. Chem. 43:2971-2974 (2000)). Earlier studies had already shown that the profound lowering of hepatic TGs, observed with a CA diet, was significantly diminished in the SHP−/− mice. In these SHP−/− mice, SREBP-1 expression was increased relative to SHP+/+ animals when both were fed a diet containing CA (Wang, et al., Dev. Cell. 2:721-731 (2002)). Another study showed that the expression of the lipogenic genes ACC, GPAT, and FAS was increased in the SHP-deficient animals (Kerr, et al., Dev. Cell. 2:713-720 (2002)). These effects with two independently derived SHP−/− lines are consistent with the direct repressive effect of SHP in SREBP-1c expression described here. This effect is also consistent with the association of hypertriglyceridemia (and mild obesity) with mutations in the SHP gene in Japanese patients (Nishigori, et al. Proc. Natl. Acad. Sci. USA 98:575-580 (2001)). We conclude that the acute attenuation of the lipogenic activity of SREBP-1c by SHP induction can explain the inhibitory effects of bile acids on TG production. Therefore, strategies aimed at increasing FXR activity and the repressive effects of SHP on SREBP-1c activity may be taken as an approach to correcting hypertriglyceridemia. The use of bile acids such as CA and CDCA is limited in humans because they can cause significant hepatotoxicity and raise LDL cholesterol by bringing about a decrease in LDL-R activity (Schoenfield, et al., Ann. Intern. Med. 95:257-282 (1981)), an effect that was also observed in our mouse studies. It might, however, be possible to develop FXR modulators that retain the beneficial effects on hepatic TG biosynthesis, but lack the LDL-raising potential of bile acids like CA and CDCA.

E. Abbreviations

The following nonstandard abbreviations have been used herein: acetyl-CoA carboxylase (ACC); acetyl-CoA synthetase (AceCS); angiopoietin-like protein 3 (ANGPTL3); carnitine palmitoyltransferase I (CPT-I); chenodeoxycholic acid (CDCA); cholesterol 7α-hydroxylase (CYP7A1); cholic acid (CA); farnesoid X receptor (FXR); fatty acid synthase (FAS); LDL receptor (LDL-R); liver receptor homolog-1 (LRH-1); liver receptor homolog-1 response element (LRH-1RE); liver X receptor (LXR); liver X receptor response element (LXRRE); long-chain acyl-CoA dehydrogenase (LCAD); malic enzyme (ME); medium-chain acyl-CoA dehydrogenase (MCAD); retinoid X receptor (RXR); short heterodimer partner (SHP); stearoyl-CoA desaturase-1 (SCD-1); triglyceride (TG).

### Example 2

### Bile acids induce energy expenditure by promoting intracellular thyroid hormone activation

The present example provides evidence that the administration of bile acids (BAs) to mice triggers energy expenditure in brown adipose tissue (BAT), resulting in weight reduction and insulin sensitization. This metabolic effect of BAs is critically dependent on the induction of the thyroid hormone activating enzyme type 2 iodothyronine deiodinase (D2) since it is lost in D2−/− mice. Human and mouse D2 expression is regulated by cAMP. BA treatment of brown adipocytes and human skeletal myocytes increases cAMP levels, D2 activity, and oxygen consumption, which is mediated via the G-protein-coupled receptor TGR5. The results suggest that the BA/cAMP/D2-T3 signaling pathway provides a target for therapeutics for improving metabolic control in human obesity and insulin resistance.

A. Introduction

In example 1, studies are discussed that suggest that the induction of SHP by BAs and its inhibitory effects on nuclear receptor signaling not only underlie the feedback regulation of BA biosynthesis, but also the downregulation of SREBP-1c-mediated hepatic fatty acid and triglyceride biosynthesis and VLDL production after BA feeding. This provides a mechanistic explanation for the reported beneficial effects of bile acids on triglyceride homeostasis (Angelin, et al., J. Lipid Res 19:1017 (1978); Bateson, et al., Br. J Clin. Pharmacol. 5:249 (1978); Carulli, et al., J. Clin. Pharmacol. 21:436 (1981); Beil, et al., Metabolism 31:438 (1982); Crouse, Am J. Med 83:243 (1987)) and suggests that BAs can function beyond the control of BA homeostasis as general metabolic integrators. In the present example, we explore the effects of BAs on energy homeostasis.

B. Materials and Methods

Materials

Isobutylmethylxanthine (IBMX), CA, CDCA, TCA, TCDCA, DCA, LCA and UDCA were obtained from Sigma (St. Quentin Fallavier, France). GW4064 was a generous gift of Shinya Inoue of Mitsubishi Pharmaceutical.

Microarray Analysis

For microarray analysis, RNA was isolated from BAT using the RNeasy mini kit (QIAGEN, Courtaboeuf, France). Equal amounts of RNA from mice in each group were pooled (n=3 or 4 mice) and used for cDNA synthesis. After second-strand cDNA synthesis (Life Technologies, Cergy Pontoise, France), in vitro transcription was performed with biotinylated UTP and CTP (Enzo Diagnostics, Farmingdale, N.Y.). Fragmented cRNA samples were hybridized to the Affymetrix MOE430A microarray. The raw data was analyzed using the Microarray Suite Software version 5.0 (MAS5.0, Affymetrix, Santa Clara, Calif.). Genes were determined to be significantly upregulated by CA or CDCA if they were commonly called Present (Detection p-value <0.05) and Increase (Change p-value <0.005) on CA and CDCA diet when compared with untreated in MAS5.0.

Cell Culture

HSMM were obtained from Cambrex (Walkersville, Md.) and were cultured according to the suppliers instructions. D2 activity measurements were performed after a 16 h incubation with the indicated compounds. For the cAMP measurements, cells were preincubated 12 h in Opti-MEM (Life Technologies, Cergy Pontoise, France), supplemented with 1 mM IBMX. Then medium was changed and cells were incubated for another 1 h in Opti-MEM with 1 mM IBMX, after which the medium was replaced for Opti-MEM with 1 mM IBMX and the indicated compounds. After 30 minutes the cells were lysed for cAMP analysis.

Oxygen Consumption and Extracellular Acidification Rate

HSMM were plated in 24 well tissue culture microplates and allowed to adhere. Quadruplicate wells were treated with nM T3, μM TCA or vehicle and incubated for 48 or 72 hours. Cells were then assayed in the CellDoctor prototype instrument from Seahorse Bioscience (Billerica, Mass.) to perform non-destructive, time resolved measurements of oxygen consumption rate and extracellular acidification rate (ECAR). This instrument uses optical sensors that profile the change in the extracellular microenvironment. Immediately prior to measurement, the medium was replaced with nonbuffered pH7.4 Specialty Media (1 part 2× DMEM and 1 part Saline) to facilitate rapid ECAR measurement. Three successive 10 min measurements were performed at 5 min intervals simultaneously in the quadriplicate wells. Immediately following measurement, total cell number was counted using a Beckman Coulter ViCell (Fullerton, Calif.).

Animal

Male C57BL/6J and KK-Ay mice, 6-7 weeks of age, were obtained from Charles River Laboratories France (l'Arbresle, France) and CLEA Japan Inc. (Tokyo, Japan), respectively. D2−/− mice were originally bred to a C57BL/6-129SV background and backcrossed for five generations to C57BL/6. All mice were maintained in a temperature-controlled (23° C.) facility with a 12 h light/dark cycle and were given free access to food and water. The control and high-fat diet was obtained from UAR (Villemoison surOrge, France). The control diet (EQ12310) contained 16.8% protein, 73.5% carbohydrate and 4.8% fat, whereas the high-fat diet (EQ/D12309) contained 23.0% protein, 35.5% carbohydrate and 35.9% fat. For treatment with BAs, mice were fed diets with 0.5% (w/w) CA or CDCA. GW4064 was suspended in peanut oil and mixed with the high fat diet at a concentration of 180 mg per kg of food. Based on their daily food intake, this resulted in a daily dose of 15 mg/kg. The mice were fasted 4 h before harvesting blood for subsequent blood measurements, and tissues for RNA isolation, lipid measurements and histology. Oxygen consumption was measured using the Oxymax apparatus (Columbus Instruments, Columbus, Ohio).

Clinical Biochemistry and Evaluation of Glucose and Lipid Homeostasis

An OGTT was performed in animals that were fasted overnight. Glucose was administered by gavage at a dose of 2 g/kg. An IPITT was done in 4 h fasted animals. Insulin was injected at a dose of 0.75 U/kg. Glucose quantification was done with the Maxi Kit Glucometer 4 (Bayer Diagnostic, Puteaux, France) or Glucose RTU (bioMérieux Inc., Marcy l'Etoile, France). Plasma insulin concentrations were measured using ELISA (Cristal Chem Inc., Downers Grove, Ill.). Nonesterified fatty acids, triglycerides, ketone bodies, BA and total cholesterol were determined by enzymatic assays (Roche, Mannheim, Germany). cAMP was measured using the cAMP-Screen system (Applied Biosystems, Bedford, Mass.).

Morphological Studies

Pieces of mouse tissues were fixed in Bouin's solution, dehydrated in ethanol, embedded in paraffin, and cut at a thickness of 5 μm. Sections were deparaffinized, rehydrated, and stained with haematoxylin and eosin. Transmission electron microscopy was then performed.

mRNA Expression Analysis by Q-RT-PCR

Expression levels were analyzed in cDNA synthesized from total mRNA using real-time PCR as described (Watanabe et al., J. Clin. Invest 113, 1408 (2004)). The sequences of the primer sets used are available at the following URL (http://www-igbmc.ustrasbg. fr/Departments/Dep_V/Dep_VA/Publi/Paper.html).

Statistical Analysis

Values were reported as mean +/− standard error. Statistical differences were determined by a Student's t test. Statistical significance is displayed as * (P <0.05) or ** (P <0.01).

C. Results and Discussion

Natural but not synthetic FXR a agonists prevent and reverse diet-induced obesity

Cholic acid (CA) has been reported to inhibit diet-induced obesity in mice (Ikemoto, et al., Am. J. Physiol. 273:37 (1997)). To characterize these metabolic effects of BAs, we studied a mouse model of diet-induced obesity in which the high fat diet was supplemented with cholic acid (CA) at 0.5% w/w.

Feeding C57BL/6J mice with a CA-containing diet for up to 47 days did not affect food intake, nutrient uptake and serum liver enzyme levels. While the mice on the high fat diet expectedly gained more weight than controls over time, supplementation of the high fat diet with CA reduced the weight gain compared to that observed in animals fed the non-supplemented chow diet. Supplementation of the chow diet with CA had no effect on body weight gain.

That the changes in weight gain reflected mainly modifications in adiposity was confirmed at necropsy, as epididymal white adipose tissue (WAT), mesenteric WAT and intrascapular BAT of the high fat-fed animals were all significantly increased in weight. The BAT of the high fat fed animals was paler, indicative of decreased metabolic activity and increased fat accumulation. In addition, there was an expansion of WAT surrounding the BAT. Diet supplementation with CA completely prevented the high fat-induced changes in adipose mass and morphology.

Remarkably, diet supplementation with CA reversed 120-days of diet-induced weight gain. When obese mice were switched to a high fat diet containing CA (F-FA), they returned to normal body weight within 30 days, an effect that was fully accounted for by a reduction in mesenteric and epididymal WAT mass. In an independent experiment, C57BL/6J mice were fed the same high fat diet supplemented with the synthetic FXRα agonist GW4064, but no protective effect against diet-induced obesity was observed. Together these results indicate that the addition of CA to a high fat diet prevents and reverses fat accumulation in a mouse model of diet-induced obesity via a mechanism that does not solely rely on FXRα.

Besides preventing and reversing diet-induced obesity, CA also prevented the development of insulin resistance. We found that CA improves glucose tolerance in C57BL/6J mice with diet-induced obesity, but that CA is able to improve insulin sensitivity in KK-Ay mouse.

Bile Acid Feeding Increases Energy Expenditure in BAT

Indirect calorimetry was used to determine whether the protective effect of CA against obesity was mediated by an increase in energy expenditure. A higher CO₂ production and O₂ consumption was evident in animals fed a high fat diet containing CA when compared to animals on a high fat or a normal diet, confirming increased energy expenditure. Feeding a high fat diet decreased the respiratory quotient (RQ=VCO₂/VO₂) indicating an increase in fat oxidation, but this was not affected by the supplementation with CA.

To identify the site(s) responsible for the increased energy expenditure, we performed a detailed histological analysis of key metabolic tissues. Liver sections of high fat fed animals showed more unstained inclusions, indicative of steatosis. When the high fat diet was supplemented with CA, the steatosis was reversed. A high fat diet also induced significant adipocyte hypertrophy in both WAT, characterized by a larger adipocyte volume, and BAT, typified by the presence of larger lipid vacuoles within the cells. This adipocyte hypertrophy was not observed when the high fat diet was supplemented with CA. Electron microscopic analysis of BAT demonstrated that the high fat diet decreased the number of mitochondrial cristae, whereas the addition of CA to the high fat diet increased the density of the mitochondria, which also showed more abundant lamellar cristae, indicating a major role for BAT in the CA-induced energy expenditure.

Molecular Mechanism of Bile Acid Action on Energy Homeostasis

To identify the molecular drivers of the effects of CA on basal metabolic rate, we performed a microarray experiment in which we compared the expression profile in liver and BAT from mice fed a high fat diet or a high fat diet mixed with CA or chenodeoxycholic acid (CDCA) for 9 days. We used also CDCA since it has similar effects as CA on energy homeostasis. As expected, major changes in hepatic gene expression were related to cholesterol and bile acid synthesis, but not to energy homeostasis.

In BAT, however, several genes involved in the control of energy expenditure were upregulated by both CA and CDCA such as the regulatory subunit 1α of phosphatidylinositol 3-kinase, forkhead box O1, glycerol kinase, nuclear receptor interacting protein 1 (RIP140) and the type 2 iodothyronine deiodinase (D2) whose expression was most affected by BA treatment. D2 converts inactive thyroxine (T4) into active 3,5,3′-triiodothyronine (T3), and is a critical determinant of thyroid hormone receptor saturation in cells in which it is expressed. The vital role of D2 in energy homeostasis is well established in mice, in which BAT adaptive thermogenesis depends on this enzyme (Bianco, et al., Endocr. Rev. 23:38 (2002)). A mouse with a targeted disruption of the D2 gene develops hypothermia when exposed to cold, and survival is only possible with a major increase in mechanical thermogenesis (shivering; de Jesus et al., J. Clin Invest 108:1379 (2001)).

We used quantitative RT-PCR to confirm the induction of D2 by BAs and to measure mRNA levels of genes involved in fatty acid metabolism, energy homeostasis and BA signaling in liver, muscle, and BAT, the primary organs involved in energy expenditure. For these studies we used organs from C57BL/6J mice fed for 47 days with chow or a high fat diet mixed with CA. The expression of peroxisome proliferator-activated receptor γ (PPARγ) coactivator-1α (PGC-1α), PGC-1β, uncoupling protein (UCP) 1, UCP-3, straight chain acyl-CoA oxidase 1 (ACO), muscle-type carnitine palmitoyltransferase I (mCPT-I), and D2 was significantly increased in BAT after BA feeding. PGC-1α and PGC-1β are transcriptional coactivators that regulate the expression of genes involved in BAT thermogenesis such as the UCPs, mitochondrial biosynthetic and fatty acid β-oxidation genes. That mRNA levels of ACO and mCPT-I were increased is not surprising, since these enzymes are involved in the β-oxidation of fatty acids, the main source of fuel for BAT mitochondria. High rates of fatty acid oxidation and adaptive heat dissipation (thermogenesis) can only be initiated and sustained in BAT due to UCPs, which dissipate the proton gradient generated during respiration across the inner mitochondrial membrane and therefore uncouples respiration from ATP synthesis. As reported previously, UCP-1 and D2 were not expressed in mouse liver and muscle under any of the conditions tested.

In liver and muscle, no major changes were detected in the expression of any of these genes. FXRα and SHP mRNA are almost not detected in the BAT when compared to the liver. In addition, none of the treatments induced the expression of these genes in BAT, whereas we could confirm the major regulation of SHP mRNA expression by BAs in liver. FGFR4 was only expressed to substantial amounts in liver. In addition, GW4064 treatment did not result in an induction of the thermogenic gene response as seen after CA treatment. These results confirm that BAT is the primary target of the metabolic effects of BAs in the mouse and confirm that the beneficial effects of BAs on energy, lipid and glucose homeostasis are not solely depending on FXRα.

D2 is essential for the bile acid-mediated prevention of diet-induced obesity

Next, we used mice with a targeted disruption of the D2 gene (D2−/− mice) and littermate controls in a diet-induced obesity study for 50 days. After chronic feeding with a high fat diet, both wildtype and D2−/− mice gained weight to a similar extent, indicating that adaptive thermogenesis was increased sufficiently in the D2−/− mice to prevent additional weight gain. This stands in contrast with the situation of acute cold-exposure, in which D2−/− BAT thermogenesis is insufficient to maintain normal body temperature (de Jesus et al., J. Clin. Invest. 108:1379 (2001)).

The addition of CA to the high fat diet prevented diet induced obesity in the wildtype mice as expected. Strikingly, CA had no effect on the development of obesity in the D2−/− mice. In these animals no changes in liver size and appearance were noted. The weight of the epididymal WAT and BAT of wildtype animals was increased upon high fat feeding and decreased when this diet was mixed with CA. However, CA was unable to prevent the increase in weight of the epididymal WAT and BAT in D2−/− mice on a high fat diet. Likewise, histological and electron microscopic analysis of BAT showed that combining the high fat diet with CA prevented lipid accumulation in BAT of wildtype but not of D2−/− mice. D2 expression was only detectable in wildtype mice and was induced upon CA feeding. Analysis of the expression of PGC-1α in BAT showed that this gene was significantly induced upon CA feeding in wildtype mice, but not in D2−/− mice, consistent with the observation that PGC-1α is regulated by thyroid hormone. Together, these results prove that D2 is an essential mediator in the prevention of diet-induced weight gain by BAs.

In vivo Effects of BA Administration

CA not only prevented and reversed diet-induced obesity in C57BL/6J mice, but also reduced obesity in a genetic diabetes model, such as the KK-Ay mice. CA addition to a normal chow diet decreased body weight due to a reduction in the WAT mass. This reduction in body weight translated into improved metabolic control as evidenced by an oral glucose tolerance test and an intraperitoneal insulin tolerance test. The effects of CA feeding on energy effects coincide, as expected, with an increase in BA pool size and a major change in BA composition. In the enterohepatic organs and serum, CA and tauroCA (TCA) and the secondary BAs of CA, deoxyCA (DCA) and TDCA, were markedly increased. The increase of DCA in serum was more pronounced when compared to the increase in the enterohepatic organs. These changes are likely to influence the different BA signaling pathways.

Since a synthetic FXRα agonist was unable to phenocopy the effects of BAs on energy expenditure, we focused our attention on FXRα-independent signaling events stimulated by BAs. In this context it is interesting to note that the expression of the D2 gene is primarily regulated via the cAMP/protein kinase A (PKA) pathway and a functional cAMP responsive element has been described in the human and mouse D2 promoter (Bianco, et al., Endocr. Rev. 23:38 (2002)). TGR5 is the only GPCR that has so far been reported to respond to BAs (Kawamata, et al., J. Biol. Chem. 278:9435 (2003); Maruyama, et al., Biochem. Biophys. Res. Commun. 298:714 (2002)). We used a cAMP response element (CRE)-luciferase reporter assay to confirm that TGR5 is a BA receptor.

The addition of both CA and DCA and their corresponding taurine conjugates (TCA and TDCA) to cells co-transfected with an expression plasmid for TGR5 and a CRE-driven luciferase reporter, increased luciferase expression in a dose-dependent manner, confirming that TGR5 responds to BAs. We used quantitative RT-PCR to measure the expression of D2 and TGR5 mRNAs in selected mouse tissues. Interestingly, D2 and TGR5 are coexpressed in mouse BAT. In contrast to D2, TGR5 expression was not changed by the addition of CA to a high fat diet. cAMP levels in BAT of mice fed a high fat diet with CA, however, were significantly elevated when compared to control mice, suggesting that the effects of BAs on BAT could be mediated via TGR5.

In vitro Effects of BA Administration

To confirm whether the effect of BAs on BAT could be reproduced in vitro, we isolated BAT cells from mice fed either a control or a high fat diet. TCA, one of the major circulating BAs, dose dependently increased D2 mRNA and activity. Interestingly, cells from high fat-fed mice were more sensitive to the addition of TCA, but also to forskolin. This could explain why BAs only have major effects on energy homeostasis when supplemented to a high fat diet and indicates that besides BAs other factors play a role in the activity of BAs. Since forskolin, which activates adenylate cyclase, has a similar effect as TCA, the effect of the high fat diet is most likely localized downstream of the GPCR.

We next tested whether cAMP could be the mediator that increases D2 expression after incubation of BAT cells with BAs. Several different BAs increased cAMP levels in BAT cells, but TCA was the most effective of the tested BAs. Interestingly, the synthetic FXRα agonist GW4064 did not increase cAMP levels in BAT cells. The effect of a high fat diet on cAMP production by BAT cells was less pronounced than on D2 mRNA levels and D2 activity. These results show that BAs can have direct systemic effects on BAT cells, resulting in increased cAMP levels, D2 mRNA and D2 activity.

In contrast to rodents, adult humans do not have significant amounts of BAT but express relatively high levels of D2 in skeletal muscle (Bianco, et al., Endocr. Rev. 23:38 (2002)), an organ of critical importance to energy homeostasis. TGR5 expression was also present in human skeletal muscle. We therefore asked whether D2 expression in human skeletal muscle could be induced by BAs in an analogy to the induction of D2 expression in BAT in mice. To address this question, we used primary cultures of human skeletal muscle myoblasts (HSMM) that express both D2 and TGR5. Upon incubation of these cells with TCA, we observed a significant and dose dependent induction of D2 activity. The synthetic FXRα agonist GW4064 once again did not induce D2 activity, confirming that the induction of D2 in HSMM is not depending on FXRα.

We next tested whether cAMP could be the mediator that increases D2 expression after incubation of HSMM with BAs. Indeed, major BA species like TCA, CA and CDCA all increased cAMP levels in a dose-dependant fashion paralleling the increase in D2 activity. Lithocholic acid (LCA), tauroCDCA (TCDCA) and GW4064, however, did not increase cellular cAMP levels.

We furthermore determined metabolic rate in HSMM after BA treatment by measuring oxygen consumption and extracellular acidification rate. Oxygen consumption is a measure for aerobic mitochondrial oxidation, whereas the extracellular acidification rate is a measure for glycolysis, lactate production and anaerobic metabolism. TCA, like T3, elevated cellular oxygen consumption in line with the induction of mitochondrial activity. In addition, we observed an increase in extracellular acidification rate for both T3 and TCA after 72 hours of treatment, which could indicate a limiting reducing capacity due to oxygen shortage. These results demonstrate that BAs can increase D2 activity and energy expenditure via a cAMP-mediated pathway in mouse BAT and HSMM.

D. Main Conclusions

We show here that in mice, dietary BA supplementation prevents and reverses diet-induced obesity by increasing energy expenditure. The major tissue mediating the effect in mice is BAT, which exhibits signs of increased adaptive thermogenesis including ultrastructural changes in mitochondrial morphology and a gene expression profile consistent with BAT activation and increased fatty acid oxidation. BA supplementation improved glucose tolerance both in a model of diet-induced obesity (C57BL/6J) and a genetic model of the metabolic syndrome (KK-Ay mice). The finding of increased D2 expression in the BAT of CA-fed animals and more importantly the loss of the CA-mediated protection against diet-induced obesity in D2−/− animals strongly indicates a major role for adaptive T3 production in BA-mediated energy expenditure. Induction of energy expenditure by this BA/cAMP/D2-T3 signaling pathway is thus identified as a target for improvement of metabolic control in obesity and insulin resistance.

The data indicate that the induction of energy expenditure in BAT by BAs is most likely independent of FXRα. First, FXRα and its target gene SHP are expressed at extremely low levels in BAT and were not induced by BAs, arguing against a major role for these nuclear receptors in BAT. Second, feeding of a high fat diet combined with the synthetic FXRα agonist GW4064 did not activate adaptive thermogenesis in BAT. Opposite to the weight reduction by BAs, GW4064 significantly increased body weight gain in mice on a high fat diet. Given the sensitivity of the D2 promoter to cAMP, FXRα-independence can be explained by the induction of D2 due to an increase in cAMP caused by binding of BAs to the GPCR TGR5.

Whereas BAT is a main site of thermogenesis in human newborns and other small mammals, adult humans lack substantial amounts of BAT (Cannon, et al., Physiol. Rev. 84:277 (2004)) and the major site for adaptive energy expenditure is skeletal muscle. It is thus quite striking that adult humans, unlike rodents, coexpress significant levels of D2 and TGR5 in skeletal muscle (Croteau, et al., J. Clin. Invest. 98:405 (1996); Salvatore, et al., Endocrinology 137:3308 (1996)). Seen in light of our findings that HSMM also coexpress TGR5 and D2 and respond to BAs by rapidly increasing cAMP levels, D2 expression, and oxygen consumption, it may be concluded that BA/cAMP/D2-T3 signaling in human skeletal muscle plays an analogous role in adaptive energy expenditure.

Several observations indirectly support such a role for D2 in determining skeletal muscle energy expenditure in humans. A recent study describes that a polymorphism in the D2 gene (Thr92A1a) is strongly associated with a decrease in glucose disposal rate that is mainly determined by muscle glucose uptake, a phenomenon that is in part dependent on muscle cell energy balance (Mentuccia, et al., Diabetes 51:880 (2002)). Also, mice lacking UCP-1 develop a pronounced compensatory increase in D2 expression not only in BAT but also in white adipose tissue (Liu et al., J. Clin. Invest. 111:399 (2003)) supporting a major role for D2 in animals that do not use UCP-1 and BAT as a major source of adaptive thermogenesis; pigs, for example, lack BAT and have been found to express D2 significantly in their skeletal muscle (Wassen et al., Endocrinology 145:4251 (2004)).

### Example 3

### GGS Protects Against Diet-Induced Obesity

The resin of the Commiphora mukul tree has been used in Ayurvedic medicine for more than 2000 years to treat a variety of ailments. Studies in both animal models and humans have shown that this resin, termed gum guggul (GGS), can decrease elevated lipid levels. The stereoisomers E- and Z-guggulsterone have been identified as the active agents in this resin.

Initial experiments with GGS in rats produced significant decreases in both LDL cholesterol and triglyceride levels. The active compounds in GGS believed to be responsible for the cholesterol-lowering properties are two steroids: E- and Z-guggulsterone. These two steroids constitute about 2 percent by weight of gum guggul. Most interestingly, GGS has been shown to be an FXR antagonist (Urizar, et al., Science 296:1703-1706 (2002); Urizar, et al., Annu. Rev. Nutr. 23:303-313 (2003)). To test the possibility that GGS also interacts with TGR5, we used CHO cells transiently expressing a TGR5 and a luciferase reporter gene driven by a cAMP-responsive element (pCRE-Luc) (Watanabe, et al., Nature 439:484-489 (2006)). It was found that treatment with different concentrations of taurocholic acid (TCA) or GGS dramatically increased reporter activity, indicating almost as much cAMP accumulation as with 10 μM forskolin.

GGS diet supplementation protects against diet-induced obesity and increases D2 activity in BAT. Mice were fed high fat diet supplemented with 1 or 2.5% GGS and it was found that this regimen conferred protection against diet-induced obesity. Since the caloric intake of mice fed a high fat diet and mice fed a high fat diet supplemented with GGS was similar, it is likely that energy expenditure is increased by GGS supplementation, a finding consistent with our previous observations when TGR5 was activated by bile acids (Watanabe, et al., Nature 439:484-489 (2006)). In these very same animals, GGS treatment promoted a significant and dose-dependent increase in BAT D2 activity (about 70-80% increase) when compared with animals fed high fat diet only. Diet supplementation with GGS was found to completely prevent the high fat diet-induced gain in adipose mass.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method of treating a human for a disease or condition selected from the group consisting of hypothyroidism; hypertriglyceridemia occurring without obesity or diabetes; thyroid dysfunction; resistance to thyroid hormone; low T3 syndrome;
Wilson's syndrome; depression; attention deficit disorder; insulin resistance occurring without diabetes or obesity; glucose intolerance occurring without diabetes or obesity; hypertension; infertility; cardiac insufficiency; Alzheimer's disease, Parkinson's disease; autism; and the aging process; said process comprising administering to said human a therapeutically effective amount of an agonist of the G protein coupled receptor TGR5.

2. The method of claim 1, wherein said agonist is a bile acid and is administered at a dosage sufficient to elevate the activity of type-2 iodothyronine deiodinase (D2) in said human's cells.

3. The method of claim 2, wherein said bile acid is selected from the group consisting of known forms of cholic acid; chenodeoxycholic acid; and tauroCA.

4. The method of claim 2, wherein said bile acid is administered orally at a dosage of between 0.1 mg/kg body weight/day and 25 mg/kg/day.

5. The method of claim 3, wherein said bile acid is administered at a dose of between 0.5 mg/kg body weight/day and 20 mg/kg/day.

6. The method of claim 1, wherein said method is a treatment for hypothyroidism.

7. The method of claim 6, wherein said agonist is a bile acid administered at between 0.1 mg/kg body weight/day and 25 mg/kg/day.

8. The method of claim 7, wherein said bile acid is administered at between 0.5 mg/kg body weight/day and 20 mg/kg/day.

9. The method of claim 8, wherein said bile acid is selected from the group consisting of: cholic acid; chenodeoxycholic acid; and tauroCA.

10. The method of claim 1, wherein said method is a treatment for Alzheimer's disease, Parkinson's disease; autism.

11. The method of claim 10, wherein said agonist is a bile acid administered at between 0.1 mg/kg body weight/day and 25 mg/kg/day.

12. The method of claim 11, wherein said bile acid is administered at between 0.5 mg/kg body weight/day and 20 mg/kg/day.

13. The method of claim 12, wherein said bile acid is selected from the group consisting of: cholic acid; chenodeoxycholic acid; and tauroCA.

14. The method of claim 1, wherein said method is a treatment for hypertriglyceridemia occurring without obesity or diabetes; resistance to thyroid hormone; low T3 syndrome; Wilson syndrome; depression; attention deficit disorder; insulin resistance occurring without diabetes or obesity; glucose intolerance occurring without diabetes or obesity; hypertension; or cardiac insufficiency.

15. The method of claim 14, wherein said agonist is a bile acid administered at between 0.1 mg/kg body weight/day and 25 mg/kg/day.

16. The method of claim 15, wherein said bile acid is administered at between 0.5 mg/kg body weight/day and 20 mg/kg/day.

17. The method of claim 16, wherein said bile acid is selected from the group consisting of: cholic acid; chenodeoxycholic acid; and tauroCA.

18. A method of assaying a test compound for use in the treatment method of claim 1, comprising:
a) incubating cells that express D2 in the presence of said test compound
b) measuring either the expression or the activity of the D2 enzyme in the incubated cells of step a);
c) comparing the activity determined in step b) with the expression or activity determined for cells in an assay conducted under similar conditions but in the absence of said test compound; and
d) concluding that said test compound should be useful in said treatment method if the activity of D2 is higher in the presence of the test compound than it in its absence.

19. A method of determining whether a test compound may be useful in the treatment method of claim 1, comprising assaying said test compound for its ability to bind to and activate the TGR5 receptor.

20. The method of claim 19, wherein said method comprises:
a) determining the ability of said test compound to bind to said TGR5 receptor wherein said TGR5 receptor is on the surface of a cell; and
b) determining whether said binding causes an increase in an intracellular activity known to be induced by the activation of TGR5.

21. The method of claim 20, wherein said intracellular activity is adenyl cyclase enzymatic activity.

22. The method of claim 20, wherein the ability of said test compound to bind to said TGR5 receptor is determined using a radioligand receptor binding assay.
